(19) United States
(12) Patent Application Publication
(10) Pub. No.: US 2023/0010933 A1
(43) Pub. Date: Jan. 12, 2023

(54) HYDROGEN PRODUCTION WITH SEQUESTRATION

(71) Applicant: G4 Insights Inc., Burnaby (CA)

(72) Inventors: Matthew Babicki, Burnaby (CA); Edson Ng, Burnaby (CA)

(73) Assignee: G4 Insights Inc., Burnaby (CA)

(21) Appl. No.: 17/946,870

(22) Filed: Sep. 16, 2022

Related U.S. Application Data
(63) Continuation of application No. PCT/CA2021/050381, filed on Mar. 23, 2021.
(60) Provisional application No. 62/993,508, filed on Mar. 23, 2020.

(51) Int. Cl. C01B 3/34; C01B 3/56; C10J 3/84

(52) U.S. Cl. CPC C01B 3/34; C01B 3/56; C10J 3/84; C01B 2203/0233; C01B 2203/0811; C01B 2203/1241; C01B 2203/042; C01B 2203/0475; C01B 2203/048; C10J 2300/0916; C10J 2300/1618; C10J 2300/0946; C10J 2300/1853

(57) ABSTRACT

Disclosed herein is a method for making hydrogen with carbon sequestration. The method may comprise using a biomass hydroconverter product to fuel a steam reformer that converts a hydrocarbon fuel stream into a gas mixture that contains at least hydrogen and carbon dioxide. The gas stream is separated to form a hydrogen-enriched gas stream and at least one hydrogen-depleted stream. The hydrogen-depleted stream may be stored or further processed to sequester the carbon contained therein. Additionally, or alternatively, the solid residue from the biomass hydroconverter also may be stored for further sequester carbon generated by the method.

### CROSS REFERENCE TO RELATED APPLICATION

This application is a continuation of International Application No. PCT/CA2021/050381, filed on Mar. 23, 2021, which claims the benefit of the earlier filing date of U.S. provisional patent application No. 62/993,508, filed Mar. 23, 2020, both of which are incorporated herein by reference in their entirety.

### FIELD

The present disclosure concerns a system and method for hydrogen production from a hydrocarbon fuel with sequestration of carbon.

### BACKGROUND

Carbon dioxide levels in the atmosphere are increasing from the use of fossil fuels for human activities. One method to minimize this increase is to utilize hydrogen as an energy carrier rather than carbon-containing fuels. Hydrogen can be made from a variety of sources. Most hydrogen is currently made from steam reforming of gaseous hydrocarbons.

Steam reforming is a commercial process used in a variety of industries. A steam reforming plant consists of a reformer (and optionally a water gas shift) section generating a mixture of gases from a hydrocarbon fuel feed, coupled with a gas separation unit creating a purified hydrogen stream (usually greater than 99% by volume) and a tail gas. The tail gas contains carbon oxides (predominantly carbon dioxide), unreformed hydrocarbons, and compounds of fuel impurities. Steam reforming is an endothermic process requiring heat, which is typically generated by combustion of a combination of additional fuel and the tail gas of the gas purification unit.

The pathway to producing the hydrogen is currently divided broadly into three categories: a) grey, from fossil fuel source, b) blue, made from fossil fuel but with carbon capture and sequestration, and c) green, manufactured from renewable sources.

The steam reforming process emits carbon dioxide in the flue of the combustion burner. The carbon dioxide comes from both the additional fuel and the process tail gas. There exist methods to capture the carbon dioxide from flue gas. Typical carbon capture systems use amine absorption. Absorption systems for carbon dioxide capture approximately 90% of the content in the flue gas. These capture systems add substantial cost and energy penalties to a reformer plant. The technology for capturing carbon dioxide from flue gas is currently much less mature than the steam reformer technology, and this blue hydrogen technology is not being implemented quickly.

The typical carbon capture system cannot capture more carbon than what is in the fossil fuel. Better systems would not only capture the fossil carbon, but also sequester carbon from the atmosphere.

There still exists the need for hydrocarbon steam reforming processes that capture carbon with lower cost and energy penalties.

### SUMMARY

Disclosed herein are embodiments of a method for producing hydrogen gas with carbon sequestration. In some embodiments, the method comprises:
i) supplying biomass to a biomass hydroconverter to form a biomass hydroconverter product and a solid residue;
ii) using the biomass hydroconverter product as a heat source fuel for a steam reformer;
iii) introducing a hydrocarbon fuel stream to the steam reformer to generate a gas mixture that contains at least hydrogen and carbon dioxide;
iv) separating the gas mixture in a gas separator to form a hydrogen-enriched gas stream and at least one hydrogen-depleted stream;
v) providing at least a first portion of the hydrogen enriched gas stream as a carbon-reduced hydrogen product; and
vi) sequestering carbon by storing the solid residue from the biomass hydroconverter process and/or storing or processing at least a first portion of the hydrogen-depleted stream.

Suitable hydrocarbon fuel streams include, but are not limited to, alcohols (for example, methanol and/or ethanol), alkanes (for example, methane, ethane, propane, and/or butane), and/or liquid fuels (such as gasoline or diesel fuel), or any combination thereof. In some embodiments, the hydrocarbon fuel stream may be, or may comprise, methane.

The method may further comprise introducing a second portion of the hydrogen enriched gas stream into the biomass hydroconverter. And/or in some embodiments, providing at least a first portion of the hydrogen enriched gas stream as a carbon-reduced hydrogen product comprises purifying the first portion of the hydrogen enriched gas stream to product a hydrogen gas stream.

In some embodiments, the biomass hydroconverter comprises a pyrolyzer and/or gasifier. In such embodiments, the biomass hydroconverter product comprises hydrocarbon volatiles, syngas, or a combination thereof, and/or the solid residue comprises biochar. And in certain embodiments, the hydrocarbon volatiles are quenched to form a bio oil.

In other embodiments, the biomass hydroconverter comprises an anaerobic digester. In such embodiments, the biomass hydroconverter product comprises methane and carbon dioxide, and/or the solid residue comprises a solid digestate.

In any embodiments, separating the gas mixture may comprise pressure swing adsorption, temperature swing adsorption, amine absorption, membrane separation, differential liquefaction, or a combination thereof. In certain embodiments, the gas separator is a pressure swing adsorption unit that optionally generates the hydrogen-enriched gas stream, a first hydrogen-depleted stream, and a second hydrogen-depleted stream. The first hydrogen-depleted stream may be a pressurized carbon dioxide stream that is at least 95% pure (on molar basis). And/or the second hydrogen-depleted stream may comprise methane and carbon dioxide, for example, at least 50% of the methane originally found in the gas mixture, such as at least 60%, at least 70%, at least 80%, at least 90% or at least 95% of the methane from the gas mixture. Additionally, or alternatively, the second hydrogen-depleted stream may be added to the hydrocarbon fuel stream that is introduced into the stream reformer.

In any embodiments, the method may further comprise providing an additional fuel material to the biomass hydroconverter, in addition to the biomass. The additional fuel material may comprise a waste material (such as, for example, plastic waste, rubber waste, refuse derived fuel, or a combination thereof), a fuel derived from biomass (such as, for example, corn sourced ethanol), a fossil derived fuel, or a combination thereof.

Additionally, or alternatively, the method may further comprise providing additional hydrogen gas to the biomass hydroconverter. In some embodiments, the additional hydrogen gas is from a renewable source.

And in any embodiments, using the biomass hydroconverter product as a heat source fuel for a steam reformer may further comprise transporting the biomass hydroconverter product to the steam reformer, such as by any suitable transportation technique. In some embodiments, the biomass hydroconverter product may be transported by mobile transport and/or a wheeling pipeline, prior to use as the heat source fuel.

The foregoing and other objects, features, and advantages of the invention will become more apparent from the following detailed description, which proceeds with reference to the accompanying FIGURES.

### BRIEF DESCRIPTION OF THE DRAWINGS

FIG. 1 is a flow chart illustrating an exemplary embodiment of the disclosed method.

### DETAILED DESCRIPTION

### I. Definitions

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. All references, including patents and patent applications cited herein, are incorporated by reference.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is recited.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

### II. Description

The steam reformer heating function can be performed with numerous energy sources. Low carbon options may include direct solar, electric heating from renewable sources, nuclear, geothermal, and others. These options either eliminate or reduce carbon dioxide emissions from the heating component of the reformer.

These options either eliminate or reduce the requirement for using fossil hydrocarbon fuel for heating value.

Biomass is defined herein as plant or animal material that can be used for energy or fuel source. Biomass can be used to generate biofuels to substitute for fossil fuels. Biomass by itself is generally not a direct substitute for fossil fuels and requires a conversion process to generate a liquid or gaseous fuel with combustion characteristics that are at least similar to hydrocarbons.

The use of biomass as the energy source for the steam reformer heating function is an alternative. In this case, the heating system does emit carbon dioxide, but the carbon comes from biomass. Carbon dioxide released from combusting fuel derived from non-fossil biomass is relatively benign, given that it simply returns to the atmosphere carbon that was recently fixed by photosynthesis.

Carbon dioxide capture of the reformer process outlet gas can also be performed. The reformer effluent gas can separate out the carbon dioxide by any one or a combination of adsorption, absorption, membrane, cryogenic, or biological processes.

A combination of utilizing low or neutral carbon heating means and reformer effluent carbon dioxide separation and capture provide an alternate method of producing blue hydrogen while using fossil hydrocarbon process input.

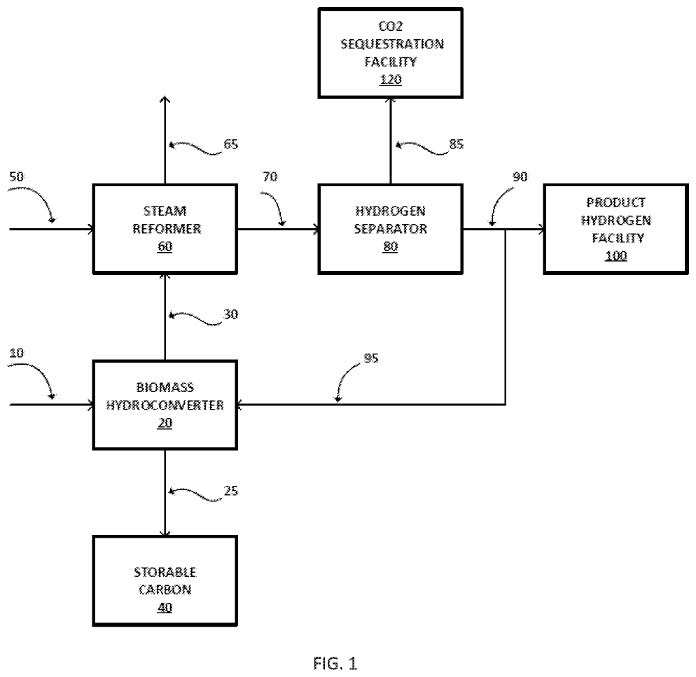

FIG. 1 shows a simplified schematic of a system or apparatus 1 according to the disclosed technology. System 1 includes a biomass hydroconverter 20, a steam reformer 60, a hydrogen separator 80, a storable carbon area 40, a product hydrogen facility 100, and a CO₂ sequestration facility 120.

Biomass 10 is prepared for the conversion process by, for example, one or more of sizing, cleaning, drying, and/or sorting steps as known in the art, so that the biomass is introduced into the biomass hydroconverter 20 in a suitable condition. The preparation steps may vary depending on individual requirements of the biomass hydroconverter 20.

The conversion process occurs in biomass hydroconverter 20. The biomass hydroconverter is used to make fuel for the steam reformer heating section burner. Methods to convert biomass into fuel use either a thermochemical or a biological process to convert at least a portion of the biomass carbon from a biological molecular structure into hydrocarbons and/or syngas. Biomass contains a significant proportion of oxygen atoms which can create fouling in the combustion process. The oxygen content is reduced in the conversion process to a hydrocarbon (in which there is no significant proportion of oxygen). In general, the conversion process comprises partitioning some of the oxygen from the produced hydrocarbon, often by coproduction of water which can then be separated from the hydrocarbon and/or syngas. Since biomass contains fewer hydrogen atoms than are typically required for the partitioning, additional hydrogen gas may be used to facilitate deoxygenation of the fuel generated by biomass. The additional hydrogen gas may be from any suitable external source, such as fossil fuels (for example, natural gas) and/or renewable sources, such as wind solar, geothermal and/or biomass sources. In certain embodiments, the additional hydrogen source is a renewable source.

One embodiment of the thermochemical method to generate reformer fuel is for the biomass to undergo pyrolysis or pyrolysis and gasification steps. The process of heating the biomass is well understood in the art. Examples include fluidized bed gasifiers, down or updraft fixed bed gasifiers, fluidized bed pyrolyzers, heated media pyrolyzers, carburizing kilns and microwave pyrolyzers. Pyrolyzers can operate from atmospheric to up to 50 barg, and operate at temperatures from 150° C. to 800° C. Gasifiers operate in the same pressure regime as pyrolyzers, but operate in temperature ranges from 600° C. to 1200° C. Pyrolysis or pyrolysis and gasification technologies can also use added materials, such as nitrogen, and heat transfer conditions to affect the outlet volatile mixture. This process can occur with or without a catalyst to assist the conversion. The pyrolysis or pyrolysis and gasification of the biomass generates a mixture of volatiles and char components. In gasification schemes, the char and volatiles are reacted into substantially a mixture of H₂O, CO, H₂, CH₄ and CO₂. Carbon residue, as unprocessed char and/or tar solids are separated and removed from the volatiles via material route 25 and generally consist of less than 10% by weight on a dry basis of the inlet biomass. After the pyrolysis or pyrolysis and gasification step, the volatiles may be converted to a hydrocarbon containing mixture over a catalyst. Injection of additional hydrogen 95 may be useful to aid in the conversion of the subsequent catalytic step.

Another embodiment is a pyrolysis to bio oil process, where the volatiles from a pyrolysis step are quenched to form a liquid mixture often referred to as bio oil. A carbon residue, typically as a solid char component, is coproduced with the volatiles and is separated from the hydroconverter via material route 25, either before or after a quenching step. The char component is generally between 10% to 30% by weight on a dry basis of the inlet biomass. Further processing of bio oil may be required to deoxygenate the oil in order for the produced fuel to combust with the lower fouling characteristics similar to hydrocarbon fuels. The deoxygenation step may be performed with hydrogen 95 over a catalyst (not shown) to form water molecules for subsequent separation from the produced fuel. Catalysts could include zeolites such as ZMS-5 or sulfided nickel-molybdenum or cobalt-molybdenum on gamma alumina.

Additionally, or alternatively, biomass hydroconverter 20 may include biological methods such as anaerobic digestion to produce methane and carbon dioxide from the biomass. This is generally performed first by acetogenic bacteria forming volatile fatty acids, and subsequently by methanogenic bacteria forming methane and carbon dioxide from the fatty acids. Injection of hydrogen into the process can be done in order to convert at least some of the resulting carbon dioxide into additional methane. This can be done with either a chemical Sabatier process or with a mixture of mesophilic and thermophilic bacteria that performs this conversion. The process also generates a carbon residue as a solid digestate, which is separated from biomass hydroconverter 20 via conveyance means 25, and generally consists of about over 75% of the mass fed into the digester. This material is generally a mixture of fertilizer and woody components and has almost 50% carbon content.

In any embodiments, biomass for hydroconverter 20 can be augmented with waste materials such as plastic or rubber waste, refuse derived fuels, and other waste materials with calorific content. The biomass can also be augmented with fuels derived from biomass such as corn sourced ethanol and such. The biomass can also be augmented with fossil derived fuel, which increases the anthropomorphic carbon content of the flue gas, but may be required in certain operational situations such as startup.

One option is where the biomass hydroconverter is located in a different location than the steam reformer. The fuel produced can be transported via pathway 30 via mobile transport such as truck or train. Another option is for produced fuel that can be injected into a pipeline that is part of a transport or distribution system. The fuel can be delivered directly or can be “wheeled” to the reformer site in an equivalent mechanism to renewable electric power.

In general, carbon collected from thermochemical conversion processes have been altered from the input materials and are relatively inert from decomposition. This form is generally known as biochar. In digesting processes, the digestate is also altered from input conditions and is also relatively inert from decomposition. The digestate can be stored in its current form, dried and stored, or heated to generate a biochar and then stored.

Solid carbon storage is well understood, benign, and low cost. Storage of wood and digestate without decomposition is possible only if the material is kept dry. Char material which has been previously pyrolyzed is able to be stored in conditions that are moist and is therefore a more stable material for long term storage. Storable carbon is defined as the remaining solids of wood, digestate or char that has been processed in the hydroconversion process. The storable carbon generally contains minerals and may also be used as a soil amendment. Storable carbon area 40 collects the solid carbon residue from biomass hydroconverter 20 via conveyance 25 and prepares the carbon for further distribution to any storage means such as terra preta, soil amendment, landfill, mixing with other materials such as concrete, or other means.

The produced fuel from the biomass hydroconverter 20 is further conveyed via pathway 30 to the burner section of steam reformer 60. The resulting flue gas 65 is emitted with CO₂ that comes from biomass and not from the hydrocarbon fuel. In one preferred option, the total amount of heat required to operate the steam reformer is provided by the biomass hydroconverter. Various heat exchange processes can be used to minimize the amount of fuel required (not shown).

After steam reforming (and optional water gas shift process, not shown), the reformed hydrocarbon fuel gas mixture is sent to a hydrogen separator 80, typically via conduit 70. Separator 80 splits the mixture to generate a hydrogen-enriched stream and at least one hydrogen-depleted stream. The separation is performed by one or more gas separating techniques such as, but not limited to, pressure swing adsorption, temperature swing adsorption, amine absorption, membrane, differential liquefaction, or other methods, or any combination of methods.

The hydrogen-enriched stream is conveyed to the product hydrogen facility 100 via conduit 90, while the hydrogen-depleted stream is conveyed to the carbon dioxide sequestration facility 120 via conduit 85. The carbon dioxide can be further separated and purified for shipment to a number of optional sequestration processes such as concrete enhancement, underground or deep ocean storage, enhanced oil recovery, other chemical binding processes or converted into useful fuel via methanation process or used in a biological enhancement process. Hydrogen-enriched stream may be further purified in hydrogen facility 100.

In a preferred option, the hydrogen separator is a pressure swing adsorption unit to generate a hydrogen-rich gas and two hydrogen-depleted streams. One potential hydrogen-depleted stream is a pressurized carbon dioxide stream that is at least 95% pure (molar basis). A second potential hydrogen-depleted stream is a pressurized stream that contains a majority of the methane and carbon monoxide contained in the reformed mixture, and optionally can be recycled back (not shown) to the reformer fuel inlet stream via conduit 50. This minimizes the export of any carbon-containing gases other than carbon dioxide.

The biomass hydroconverter 20 is optionally fed at least a portion of the hydrogen-enriched stream via conduit 95. The hydrogen in this stream is utilized in the hydroconverter 20 with the biomass 10 to generate the burner fuel in pathway 30 and the solid carbon-containing material sent to storage carbon facility 40. The hydrogen for hydroconverter 20 can be augmented with hydrogen from external sources such as green hydrogen or gray hydrogen as required for process conditions.

The carbon residue from the cases above can be collected and stored. By storing at least a portion of the carbon-containing residue generated in the biomass hydroconverter, a net sequestration of CO₂ is achieved while concurrently producing biomass-derived hydrocarbon fuel.

CO₂ storage is not yet a mature technology and there are considerable outstanding issues regarding the longevity, safety, legal and regulatory framework, and costs associated with it.

The profitability for biofuel production can be positive depending mostly on the price difference between the value of the fuel and the biomass costs. This allows the CO₂ sequestration to be subsidized by the fuel production and reducing or eliminating the net cost of sequestration.

In many methods, carbon residue is used as energy source for the biofuel production process. With the use of externally supplied hydrogen from renewable sources, the biofuel process can reduce or eliminate the need for using carbon remnant as an energy source. In this manner, the energy source is substituted by energy from intermittent renewable sources.

### III. Examples

A steam reformer using methane input of 390 kg/hr is reformed and produces 120 kg/hr of hydrogen at 99.5% purity. The reformer is heated by a burner using a portion of the inlet methane and the tail gas from a pressure swing adsorption hydrogen purification unit. An amine system collects 970 kg/hr of carbon dioxide from the flue gas, allowing 105 kg/hr of remaining carbon dioxide to be emitted.

The carbon intensity is calculated at +6 gCO₂e/MJ of hydrogen. Carbon intensity is calculated as the ratio of mass of CO₂ (equivalent) emitted divided by higher heating value of hydrogen mass exported. In this case carbon intensity equals 105 kg CO₂ equivalent per 120 kg hydrogen times 144 MJ per kg.

In contrast using an embodiment of the disclosed method, for the same hydrogen output, the steam reformer uses a methane input of 330 kg/hr, and stored carbon dioxide is at 910 kg/hr. The biomass hydroconverter, using a portion of the generated hydrogen, requires a feed rate 10 tonnes/day of oven dry biomass, and storable carbon rate is 75 kg/hr. The carbon intensity is calculated at negative 12 gCO₂e/MJ of hydrogen.

In some embodiments of the disclosed method, the carbon intensity is less than zero, such as from less than zero to −20 gCO₂e/MJ or more negative.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A method, comprising:
i) supplying biomass to a biomass hydroconverter to form a biomass hydroconverter product and a solid residue;
ii) using the biomass hydroconverter product as a heat source fuel for a steam reformer;
iii) introducing a hydrocarbon fuel stream to the steam reformer to generate a gas mixture that contains at least hydrogen and carbon dioxide;
iv) separating the gas mixture in a gas separator to form a hydrogen-enriched gas stream and at least one hydrogen-depleted stream;
v) providing at least a first portion of the hydrogen enriched gas stream as a carbon-reduced hydrogen product; and
vi) sequestering carbon by storing the solid residue from the biomass hydroconverter process and/or storing or processing at least a first portion of the hydrogen-depleted stream.

2. The method of claim 1, comprising introducing a second portion of the hydrogen enriched gas stream into the biomass hydroconverter.

3. The method of claim 1, wherein providing at least a first portion of the hydrogen enriched gas stream as a carbon-reduced hydrogen product comprises purifying the first portion of the hydrogen enriched gas stream to produce a hydrogen gas stream.

4. The method of claim 1, wherein the biomass hydroconverter comprises a pyrolyzer and/or gasifier, the biomass hydroconverter product comprises hydrocarbon volatiles, syngas, or a combination thereof, and the solid residue comprises biochar.

5. The method of claim 4, wherein the hydrocarbon volatiles are quenched to form bio oil.

6. The method of claim 1, wherein the biomass hydroconverter comprises an anaerobic digester, the biomass hydroconverter product comprises methane and carbon dioxide, and the solid residue comprises a solid digestate.

7. The method of claim 1, wherein separating the gas mixture comprises pressure swing adsorption, temperature swing adsorption, amine absorption, membrane separation, differential liquefaction, or a combination thereof.

8. The method of claim 1, wherein the gas separator is a pressure swing adsorption unit that generates the hydrogen-enriched gas stream and a first and a second hydrogen-depleted stream.

9. The method of claim 8, wherein the first hydrogen-depleted stream is a pressurized carbon dioxide stream that is at least 95% pure (molar basis).

10. The method of claim 8, wherein the second hydrogen-depleted stream comprises methane and carbon dioxide.

11. The method of claim 10, wherein the second hydrogen-depleted stream is added to the hydrocarbon fuel stream that is introduced into the steam reformer.

12. The method of claim 1, wherein the hydrocarbon fuel is an alkane fuel, an alcohol fuel, a liquid fuel, or a combination thereof.

13. The method of claim 12, wherein:
the alkane fuel is methane, ethane, propane, butane, or a combination thereof,
the alcohol fuel is methanol, ethanol, or a combination thereof; and
the liquid fuel is gasoline, diesel fuel or a combination thereof.

14. The method of claim 1, wherein the hydrocarbon fuel stream is or comprises methane.

15. The method of claim 1, further comprising providing an additional fuel material to the biomass hydroconverter, in addition to the biomass.

16. The method of claim 15, wherein the additional fuel material comprises a waste material, a fuel derived from biomass, a fossil derived fuel, or a combination thereof.

17. The method of claim 16, wherein:
the water material comprises plastic waste, rubber waste, refuse derived fuel, or a combination thereof, and
the fuel derived from biomass comprises corn sourced ethanol.

18. The method of claim 1, wherein using the biomass hydroconverter product as a heat source fuel for a steam reformer comprises transporting the biomass hydroconverter product to the steam reformer by mobile transport and/or a wheeling pipeline, prior to using the product as a heat source fuel.

19. The method of claim 1, further comprising providing additional hydrogen gas to the biomass hydroconverter.

20. The method of claim 19, wherein the additional hydrogen gas is from a renewable source.
